(19) United States
(12) Patent Application Publication
(10) Pub. No.: US 2007/0015943 A1
(43) Pub. Date: Jan. 18, 2007

(54) Method of preparation of optically active alcohols

(75) Inventors: Mahn-Joo Kim, Pohang (KR); Jaiwook Park, Pohang (KR); Yong II Chung, Pohang (KR); Jun Ho Choi, Pohang (KR); Han Ki Lee, Busan (KR); Yoon Kyung Choi, Gyeongju (KR); Daeho Kim, Gyeonjgsangnam-do (KR)

Correspondence Address: MARSHALL, GERSTEIN & BORUN LLP, 233 S. WACKER DRIVE, SUITE 6300, SEARS TOWER, CHICAGO, IL, 60606, US

(73) Assignees: POSTECH FOUNDATION, Pohang (KR); POSCO, Pohang (KR)

(21) Appl. No.: 10/565,829

(22) PCT Filed: Jul. 25, 2003
(86) PCT No.: PCT/KR03/01494

§ 371 Date: Aug. 25, 2006

(51) Int. Cl. C12P 7/62; C12P 7/02

(52) U.S. Cl. 568/807; 435/135; 435/155

(57) ABSTRACT

The present invention relates to a method for preparing chiral alcohol having optical activity. More specifically, the present invention relates to a method for preparing (S)-chiral alcohol with a high yield and a high optical purity by mixing achiral substrates such as racemic alcohol or ketone with metal catalyst and protein hydrolase to perform a dynamic kinetic resolution reaction.

### FIELD OF THE INVENTION

The present invention relates to a method of preparing a chiral alcohol with optical activity, and more particularly, to a method of preparing a chiral alcohol with optical activity and high optical purity by using metal catalyst and enzyme catalyst in one reaction vessel.

### BACKGROUND OF THE INVENTION

A method for steroselective synthesis of one enantiomer is an important tool in synthetic chemistry. Especially, since optically active alcohols are important in asymmetric synthesis, the presentation of stereoselective synthesis of an optically pure alcohol is very important.

Conventional stereoselective syntheses of optically active alcohol include a method of synthesizing the alcohol using chiral metal catalyst or ligand and a method of performing optical resolution using enzyme. However, the chiral metal catalyst or ligand is very costly and the method of kinetic resolution has a low yield of less than 50%.

In order to overcome the above shortcomings, a dynamic kinetic resolution (DKR) by the combination of enzyme catalyst and metal catalyst has been suggested (Persson, B. A.; Larsson, A. L. E.; Ray, M. L.; Baeckvall, J.-E. J. Am. Chem. Soc. 1999, 121, 1645.; Lee, D. H.; Huh, E. A.; Kim, M.-J.; Jung, H. M.; Koh, J. H.; Park, J. Org. Lett. 2000, 2, 2377.; Choi, J. H.; Kim, Y. H.; Nam, S. H.; Shin, S. T.; Kim, M.-J.; Park, J. Angew. Chem. Int. Ed. 2002, 41, 2373.).

The above method uses both enzyme catalyst and metal catalyst and thus does not need chiral ligand. The method is effective asymmetric synthesis in that it can overcome the limitations of the previous simple kinetic resolution method. However, since it uses lipase as enzyme catalyst, only ®-enantiomer can be synthesized. That is to say, in the case of 1-phenylethanol, only an ®-chiral alcohol can be synthesized and an (S)-chiral alcohol is not obtained.

However, (S)-chiral alcohol which is counter enantiomer synthesized using lipase is also an important optical enantiomer in asymmetric synthesis in the field of fine chemistry where pharmaceutical drugs, pesticides, cosmetics, food additives and so on are synthesized. Therefore, a selective synthesis method of such an (S)-enantiomer has been seriously needed. However, up to now a synthesis method of (S)-chiral alcohols with high optical purity and high yield has not been suggested.

### DETAILED DESCRIPTION OF THE INVENTION

The present invention provides a method of synthesizing (S)-chiral alcohol enantioselectively with a high optical purity and a high yield. The (S)-chiral alcohol is an counter enantiomer of a chiral alcohol which can be obtained using lipase in the conventional dynamic kinetic resolution method.

In order to attain the above aspect and other aspects, the present invention provides a method of preparing (S)-chiral alcohol. The method includes:

(a) reacting in organic solvent a compound of a following chemical formula 1 as a starting material,

a racemization metal catalyst,

an acyl donor being capable of acylating an alcohol compound, and

a protein hydrolysis enzyme being capable of stimulating the enantioselective acylation of a racemic compound to obtain a chiral ester compound of chemical formula 3; and

(b) hydrolyzing the chiral ester compound of chemical formula 3 to obtain (s)-chiral alcohol.

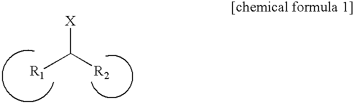

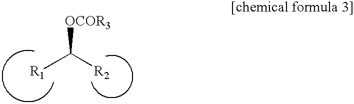

where X is —OH or ═O,

R₁, R₂ and R₃ are independently substituted or unsubstituted C₁-C₁₅ alkyls, substituted or unsubstituted C₂-C₁₅ alkenyls, substituted or unsubstituted C₂-C₁₅ alkynyls, substituted or unsubstituted C₅-C₁₈ aryls, substituted or unsubstituted C₆-C₁₈ arylalkyls, substituted or unsubstituted C₂-C₂₀ heterocycles, substituted or unsubstituted C₃-C₂₀ heteroarylalkyls, substituted or unsubstituted C₃-C₁₅ cycloalkyls, substituted or unsubstituted C₃-C₁₅ cycloalkenyls, substituted or unsubstituted C₆-C₁₅ cycloalkynyls, or substituted or unsubstituted C₃-C₂₀ heterocycloalkyls, and R₁ and R₂ can be linked together. R₁ and R₂ may be linked together to form, specifically, a substituted or unsubstituted C₇-C₂₀ fused ring or a substituted or unsubstituted C₅-C₂₀ hetero fused ring.

In the above formulas, a size of a circular arc may indicate that R₁ group is larger than R₂ group.

In the preparation method, when a starting material is a compound having a chemical formula 1 such as a ketone where X is ═O, a hydrogen donor may be added in the (a) step.

The preparation is described in two cases: when the compound of chemical formula 1 is the compound of the chemical formula 1a having an alcohol group and when the compound of chemical formula 1 is the compound of the chemical formula 1b having a ketone group.

In a case where the compound of chemical formula 1 is the compound of the chemical formula 1a, the method includes:

(a) reacting in organic solvent the compound of the following chemical formula 1a;

a racemization metal catalyst,

an acyl donor being capable of acylating an alcohol compound, and

a protein hydrolysis enzyme being capable of stimulating the enantioselective acylation of a racemic compound to obtain a chiral ester compound of chemical formula 3; and

(b) hydrolyzing the chiral ester compound of chemical formula 3 to obtain an (s)-chiral alcohol.

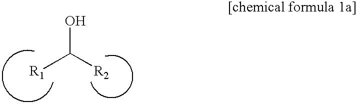

where, R₁ and R₂ are the same as defined in chemical formula 1.

In the case where a compound of chemical formula 1 is the compound of the chemical formula 1b, the method includes:

(a) reacting in organic solvent the compound of the following chemical formula 1b, a racemization metal catalyst,

a hydrogen donor being capable of reducing a ketone to an alcohol,

an acyl donor being capable of acylating an alcohol compound, and

a protein hydrolysis enzyme being capable of stimulating the enantioselective acylation of a racemic compound to obtain a chiral ester compound of chemical formula 3; and

(b) hydrolyzing the chiral ester compound of chemical formula 3 to obtain an (s)-chiral alcohol.

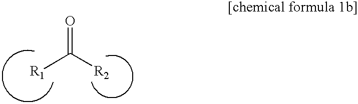

R₁ and R₂ may defined as defined above in chemical formula 1.

The preparation method of the present invention has representative features as follows: an (S)-chiral alcohol which is impossible to prepare using lipase in conventional dynamic kinetic resolution method can be obtained by using a protein hydrolysis enzyme instead of the lipase in (a) step. Step (a) may be a one-pot reaction which is performed in one reaction vessel.

In step (a), the compound of chemical formula 1 is used as a substrate in an organic solvent, and dynamic kinetic resolution is performed by the combination of metal catalyst and enzyme catalyst, protein hydrolysis enzyme, in one reaction vessel to obtain an (S)-chiral ester having optical activity. The reaction described in step (a) is a one-pot reaction where all the reaction materials react simultaneously without separation of reaction intermediates. When the substrate is a compound of chemical formula 1b having a ketone group, a hydrogen donor is added, and thus the ketone group is reduced to an alcohol group before the above described reaction. This reaction is also one-pot reaction where all reactions after the reduction are performed simultaneously.

An (S)-chiral ester prepared in step (a) is converted to an (S)-chiral alcohol by conventional hydrolysis.

The preparation of a chiral compound of chemical formula 3 is described in more detail.

The following compounds are mixed in a solvent to prepare a chiral compound having chemical formula 3: a substrate including a compound having chemical formula 1 with either an alcohol or a ketone group; metal catalyst which stimulates a reduction reaction of the ketone to an alcohol when the compound of chemical formula 1 has a ketone group, and stimulates racemization reaction of an alcohol; hydrogen donor for reducing ketone group when the compound of chemical formula 1 has ketone group; acyl donor being capable of acylating an alcohol compound of chemical formula 1; and protein hydrolysis enzyme being capable of leading the enantioselective acylation of one enantiomer of racemic alcohols.

The resulting mixture is purged with inert gas to romove oxygen, and is agitated at 0° C. to 100° C., preferably at room temperature to 80° C. to finish the reaction. Subsequently, the reaction mixture is worked up, and purified to obtain chiral compound of chemical formula 3.

In the above reaction, the acyl donor is a compound of the following chemical formula 2. However, acyl donor of chemical formula 2 need not be added additionally, when the compound of chemical formula 1 includes an acyl donor.

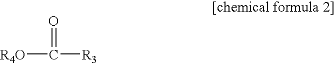

where R₃ and R₄ are independently substituted or unsubstituted C₁-C₁₅ alkyls, substituted or unsubstituted C₂-C₁₅ alkenyls, substituted or unsubstituted C₂-C₁₅ alkynyls, substituted or unsubstituted C₅-C₁₈ aryls, substituted or unsubstituted C₆-C₁₈ arylalkyls, substituted or unsubstituted C₂-C₂₀ heterocycles, substituted or unsubstituted C₃-C₂₀ heteroarylalkyls, substituted or unsubstituted C₃-C₁₅ cycloalkyls, substituted or unsubstituted C₃-C₁₅ cycloalkenyls, substituted or unsubstituted C₆-C₁₅ cycloalkynyls, or substituted or unsubstituted C₃-C₂₀ heterocycloalkyls.

When a compound of chemical formula 1 includes an acyl donor, R₁ or R₂ may include a substituent having an —OCO—R₃ terminal group. Some compounds having a structure as described by chemical formula 1, for example 3-(1-hydroxyethyl)phenyl butyrate, do not need a separate addition of an acyl donor.

As described above, the metal catalyst stimulates the reduction of a compound having a structure described by chemical formula 1 and the conversion into a racemic compound. The metal catalyst includes a ruthenium complex compound, preferably ruthenium complex compound as depicted in chemical formulas 4-8 below.

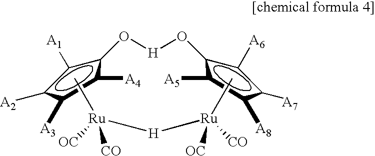

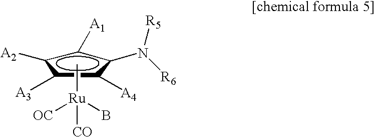

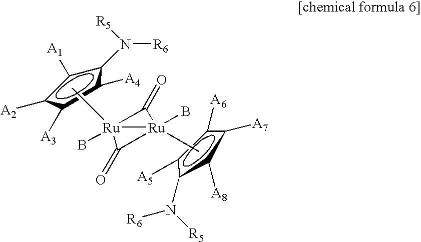

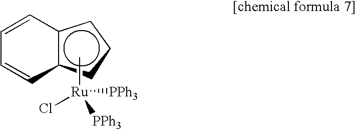

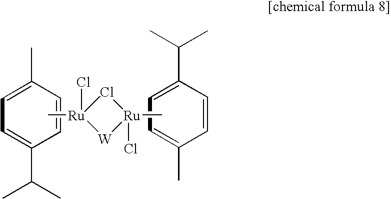

where, A₁, A₂, A₃, A₄, A₅, A₆, A₇ and A₈ may be hydrogen, substituted or unsubstituted C₁-C₁₀ alkyls, substituted or unsubstituted C₅-C₁₈ aryls, or substituted or unsubstituted C₂-C₂₀ heterocycles.

R₅ and R₆ may be hydrogen, substituted or unsubstituted C₁-C₁₅ alkyls, substituted or unsubstituted C₂-C₁₅ alkenyls, substituted or unsubstituted C₂-C₁₅ alkynyls, substituted or unsubstituted C₅-C₁₈ aryls, substituted or unsubstituted C₆-C₁₈ arylalkyls, substituted or unsubstituted C₂-C₂₀ heterocycles, substituted or unsubstituted C₃-C₂₀ heteroarylalkyls, substituted or unsubstituted C₃-C₁₅ cycloalkyls, substituted or unsubstituted C₃-C₁₅ cycloalkenyls, substituted or unsubstituted C₆-C₁₅ cycloalkynyls, or substituted or unsubstituted C₃-C₂₀ heterocycloalkyls.

B is a substituent selected from the group consisting of hydrogen, carbonyl, halogen and trifluoromethanesulfonate (herein referred to as —OTf). In some embodiments, there may be no substituent at the B site.

W is a hydrogen or a halogen.

In the above chemical formulas, examples of unsubstituted C₁-C₁₅ alkyl may include methyl, ethyl, propyl, isopropyl, isobutyl, sec-butyl, pentyl, iso-amyl, hexyl and so on. At least one of the alkyls can be substituted for using halogen, hydroxy, nitro, cyano, amino, azido, amido, hydrazine, hydrazone, ester, carboxyl or salt thereof, sulfonic acid or salt thereof, phosphoric acid or salt thereof, or a C₁-C₁₅ alkyl, alkenyl, alkynyl, cycloalkyl, cycloalkenyl, cycloalkynyl, C₁-C₁₆ heteroalkyl, C₅-C₁₈ aryl, C₆-C₁₈ arylalkyl, C₂-C₂₀ heterocycle, or C₃-C₂₀ heteroarylalkyl.

The unsubstituted C₂-C₁₅ alkenyl or alkynyl may include a carbon double or a triple bond at an intermediate site or a terminal site of the alkyl as defined above. Specific examples include vinyl, propenyl, butenyl, hexenyl, ethynyl and so on. At least one hydrogen on the alkenyl or the alkynyl can be substituted for using halogen, hydroxy, nitro, cyano, amino, azido, amido, hydrazine, hydrazone, ester, carboxyl or a salt thereof, sulfonic acid or a salt thereof, phosphoric acid or a salt thereof, or a C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The heteroalkyl may include nitrogen, sulfur, oxygen or phosphorus. Specific examples include methoxy, ethoxy, propoxy, isopropoxy, butoxy, sec-butoxy, t-butoxy, benzyloxy, naphthyloxy and triphenylmethoxy. Examples having substituents include a haloalkoxy radical such as fluoromethoxy, chloromethoxy, trifluoromethoxy, trifluoroethoxy, fluoroethoxy and fluoropropoxy. At least one hydrogen of a heteroalkyl can be substituted for using halogen, hydroxy, nitro, cyano, amino, azido, amido, hydrazine, hydrazone, ester, carboxyl or salt thereof, sulfonic acid or salt thereof, phosphoric acid or salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The aryl may include a C₅-C₁₈ carbocyclic aromatic group may form a single ring or a combination of rings. The ring can be attached as a pendent group or can be fused. The term of aryl may include an aromatic radical such as phenyl, naphthyl, tetrahydronaphthyl, indane, cyclopentadienyl and biphenyl. The aryl can have at least one substituent such as hydroxyl, halo, haloalkyl, nitro, cyano, alkyl, alkoxy and low alkylamino. At least one hydrogen of aryl can be substituted for using halogen, hydroxy, nitro, cyano, amino, azido, amido, hydrazine, hydrazone, ester, carboxyl or a salt thereof, sulfonic acid or a salt thereof, phosphoric acid or a salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The arylalkyl may be defined as a compound where at least one hydrogen is substituted for using a low alkyl radical, for example methyl, ethyl, propyl and so on. Specific examples may include benzyl, phenylethyl and so on. At least one hydrogen of an arylalkyl can be substituted for using halogen, hydroxy, nitro, cyano, amino, azido, amido, hydrazine, hydrazone, ester, carboxyl or salt thereof, sulfonic acid or salt thereof, phosphoric acid or salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The heterocycle may include 4 to 20 atoms of a cyclic radical including 1, 2 or 3 heteroatoms selected from a group consisting of N, O, P and S. In some embodiments, the remaining atoms may be carbon. The term also refers to a cyclic aromatic radical where heteroatoms in a ring formation are oxidized or become quaternary to form for example N-oxide or a quaternary salt. Specific examples may include, but are not limited to thienyl, puryl, benzothienyl, pyridyl, prazinyl, pyrimidinyl, pyridazinyl, quinolinyl, quinoxalinyl, imidazolyl, puranyl, benzopuranyl, thiazolyl, isoxazolyl, benzisoxazolyl, benzimidazolyl, triazolyl, pyrazolyl, pyrrolyl, indolyl, pyridonyl, N-alkyl-2-pyridonyl, pyrazinonyl, pyridazinonyl, pyrimidinonyl, oxazolonyl and N-oxide thereof (for example, pyridyl N-oxide, quinolinyl N-oxide), quaternary salt thereof. At least one hydrogen of the heteroatoms can be substituted for using halogen, hydroxy, nitro, cyano, amino, azido, amido, hydrazine, hydrazone, ester, carboxyl or salt thereof, sulfonic acid or salt thereof, phosphoric acid or salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The heteroarylalkyl is one where hydrogens may be substituted for using alkyl. At least one hydrogen of the heteroarylalkyl can be substituted for using halogen, hydroxy, nitro, cyano, amino, azido, amido, hydrazine, hydrazone, ester, carboxyl or a salt thereof, sulfonic acid or a salt thereof, phosphoric acid or a salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The cycloalkyl and cycloalkenyl may be a C₃-C₁₅ cyclic radical. At least one hydrogen of the cycloalkyl and cycloalkenyl can be substituted for using halogen, hydroxy, nitro, cyano, amino, azido, amido, hydrazine, hydrazone, ester, carboxyl or salt thereof, sulfonic acid or salt thereof, phosphoric acid or salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The cycloalkynyl is a C₆-C₁₅ cyclic radical. At least one hydrogen of the cycloalkynyl can be substituted for using halogen, hydroxy, nitro, cyano, amino, azido, amido, hydrazine, hydrazone, ester, carboxyl or a salt thereof, sulfonic acid or a salt thereof, phosphoric acid or a salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The heterocycloalkyl may include 4 to 20 atoms of a cyclic radical including 1, 2 or 3 heteroatoms selected from a group consisting of N, O, P and S, and the remaining atoms may be carbon. That is to say, hydrogens of the cycloalkyl may be substituted for using an alkyl and heteroatom is included. At least one hydrogen of heterocycloalkyl can be substituted for using halogen, hydroxy, nitro, cyano, amino, azido, amido, hydrazine, hydrazone, ester, carboxyl or salt thereof, sulfonic acid or salt thereof, phosphoric acid or salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The fused ring may include 7 to 20 atoms in a bicyclic or tricyclic aromatic radical where R₁ and R₂ are linked to form a ring and aryl ring which may be substituted. For example, specific examples include indanyl, indenyl, dihydronaphthyl, tetrahydronaphthyl etc. At least one hydrogen of the fused ring can be substituted for using halogen, hydroxy, nitro, cyano, amino, azido, amido, hydrazine, hydrazone, ester, carboxyl or a salt thereof, sulfonic acid or a salt thereof, phosphoric acid or a salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The hetero fused ring may include 6 to 20 atoms in a bicyclic or tricyclic radical including 1, 2 or 3 heteroatoms selected from a group consisting of N, O, P and S, with the remaining atoms in the radical being carbon. The term also means cyclic aromatic radical where heteroatoms in the ring are oxidized or become quaternary to form, for example, an N-oxide or a quaternary salt. Specific examples may include, but are not limited to benzothienyl, cumaryl, quinolinyl, quinoxalinyl, benzopuranyl, benzothiazolyl, benzoisoxazolyl, benzoimidazolyl, indolyl, benzopyridonyl, N-alkyl-2-benzopyridonyl, benzopyrazinonyl, benzopyridazinonyl, benzopyrimidinonyl, benzooxazolonyl, an N-oxide (for example, pyridyl N-oxide, quinoliny N-oxide), or a quaternary salt. At least one hydrogen of the heteroatoms can be substituted for using halogen, hydroxy, nitro, cyano, amino, azido, amido, hydrazine, hydrazone, ester, carboxyl or a salt thereof, sulfonic acid or a salt thereof, phosphoric acid or a salt thereof, or C₁-C₁₅ alkyl, alkenyl, alkynyl, cycloalkyl, cycloalkenyl, cycloalkynyl, C₁-C₁₅ heteroalkyl, C₅-C₁₈ aryl, C₆-C₁₈ arylalkyl, C₂-C₂₀ heterocycle, or C₃-C₂₀ heteroarylalkyl.

The protein hydrolysis enzyme plays a role in acylating an alcohol enantioselectively in organic solvent in the presence of an acyl donor. The protein hydrolysis enzyme stimulates the stereoselective acylation of an (S)-chiral compound of racemic compounds which is racemized by the metal catalyst. Exemplary protein hydrolysis enzymes may include, but are not limited to stabilized or fixed subtilisin, chymotrypsin, papain, protease from Aspergillus orygae, protease from Aspergillus melleus, protease from Streptomyces griseus, protease from Bacillus stearothemophilus, etc. Among the protein hydrolysis enzymes, a protein hydrolysis enzyme with opposite stereoelectivity to lipase, or a lipid hydrolysis enzyme with respect to secondary alcohol can be used in the present invention. An example of a useful protein hydrolysis enzyme with opposite stereoelectivity to lipase is subtilisin. Commercially available stabilized subtilisin includes subtilisin-CLEC. When it is necessary, subtilisin is stabilized in aqueous pyridine solution using polyether-based sufactant. The protein hydrolysis enzymes can be used in an amount of 5 to 1000 mg per 1 mmol of reactive substrate, especially 10 to 300 mg per 1 mmol of reactive substrate.

A hydrogen donor reduces a ketone group of compound having a structure of chemical formula 1 to an alcohol group in the presence of a metal catalyst. Hydrogen donors may include, but are not limited to 2,4-dimethyl-3-pentanol, 2,6-dimethyl-4-heptanol, formic acid, hydrogen. In order to remove the hydrogen easily after production of a chiral ester, it is preferable to use the hydrogen donor under normal pressure. The hydrogen donor is preferably used in an amount of 1 to 10 moles on the basis of 1 mole of the compound having a structure of chemical formula 1.

In some embodiments, since the enzyme catalyst reaction (e.g. protein hydrolysis enzyme) has been affected by solvent in terms of synthesis yield of product and enantioselectivity, the following solvents are preferred: aprotic solvent selected from benzene; toluene; C₅-C₁₀ alkane; C₅-C₁₀ cycloalkane; tetrahydrofuran; dioxane; C₂-C₁₀ dialkylether such as ethylether, diisopropyl ether or t-butyl methylether; C₃-C₁₀ alkylate such as ethyl acetate, propyl acetate or ethyl propionate; C₂-C₁₀ cyanoalkane such as acetonitrile or propionitrile; C₃-C₁₀ dialkyl ketone such as acetone or methylethyl ketone; dichloromethane; chloroform; carbon tetrachloride, or C₄-C₁₀ tertiary alcohol having high hydrophobicity such as tert-butanol or 3-methyl-3-pentanol. Additionally, a room temperature ionic liquid such as 1-methyl-3-ethylimidazolium tetrafluoroborate or 1-methyl-3-butylimidazolium hexafluorophosphate can be also used. In some embodiments, the solvent is preferably controlled so that the concentration of dissolved solute is in a range from 0.1 to 0.8M.

The reaction temperature of dynamic kinetic resolution depends on the kind of the reaction materials and is preferably in a range from 0 to 100° C. In some embodiments, the reaction temperature may be in a range from room temperature to 80° C. When the reaction temperature is less than room temperature, a reaction rate is slow and when it is more than 80° C., the enzyme loses its activity.

Through the reaction outline in step (a), an (S)-chiral ester compound of chemical formula 3 is prepared.

### EXAMPLE

### Example 1

To a Schlenk flask, 3.7 mg of (Ph₄C₅NHCHMe₂)Ru(CO)₂Cl and 18 μL of t-BuOK solution (1 M in THF) was added and dried under the reduced pressure. 1 mL of toluene was added and then agitated for 1 hour. After the toluene was removed under the reduced pressure, 9 mg of stabilized subtilisin, 31.8 mg of sodium carbonate, 18 μL of 1-phenylethanol, 39 μL of 2,2,2-trifluoroethylbutyrate and 0.5 mL of THF were added, The mixture was agitated at room temperature for three days. After termination of the reaction, catalyst was filtered, the obtained filtrated solution was concentrated and separated using column chromatography (silica gel, ethyl acetate/hexane=4:1). Optical purity of the product was measured using high resolution liquid chromatography equipped with a chiral column. The yield of the produced (S)-acetate was 95% and optical purity was 92% enantiomeric excess (herein referred to as ee). (S)-alcohol was obtained by adding (S)-acetate and 2 equivalents of K₂CO₃ to 80% methanol solution and hydrolyzing at room temperature.

[α]²⁵_{D}=−87.3 (c=1.01, CHCl₃);

¹H NMR (300 MHz, CDCl₃, ppm) 7.35-7.28 (m, 5H), 5.90 (q, J=6.6 Hz, 1H), 2.31 (t, J=7.4 Hz, 2H), 1.68-1.58 (m, 2H), 1.53 (d, J=6.6 Hz, 3H), 0.92 (t, J=7.4 Hz, 3H).

### Example 2

To a Schlenk flask, 5.9 mg of (η⁵-Ph₄C₄CO)₂H(μ-H)(CO)₄Ru₂, 16 mg of stabilized subtilisin, 43 mg of 1-p-chlorophenylethanol, 39 μL of 4-chlorophenylbutyrate and 1 mL of toluene were added and then agitated at 60° C. for three days. After termination of the reaction, the catalyst was filtered, the obtained filtrated solution was concentrated and the product was separated using column chromatography (silica gel, ethyl acetate/hexane=4:1). Optical purity of the product was measured using high resolution liquid chromatography equipped with a chiral column. The yield of the produced (S)-acetate was 92% and optical purity was 99% ee. The chiral acetate was hydrolyzed using a basic aqueous alcoholic solution and was converted to the corresponding chiral alcohol.

[α]²⁵_{D}=−96 (c=1.03, CHCl₃);

¹H NMR (300 MHz, CDCl₃, ppm) 7.33-7.29 (m, 4H), 5.85 (q, J=6.6 Hz, 1H), 2.30 (t, J=7.4 Hz, 2H), 1.68-1.58 (m, 2H), 1.50 (d, J=6.6 Hz, 3H), 0.92 (t, J=7.4 Hz, 3H).

### Example 3

To a well-dried Schlenk flask, 7.4 mg of (Ph₄C₅NHCHMe₂)Ru(CO)₂Cl and 36 μL of t-BuOK solution (1 M in THF) was added and dried under the reduced pressure. 0.5 mL of toluene was added and then agitated for 1 hour. After the toluene was removed under the reduced pressure, 18 mg of subtilisin-CLEC, 62.6 mg of sodium carbonate, 43 mg of 1-p-methoxyphenylethanol, 39 μL of 4-chlorophenyl butyrate and 0.5 mL of THF were added, The mixture was agitated at room temperature for three days. After termination of the reaction, the catalyst was filtered, the obtained filtrated solution was concentrated. The product was separated using column chromatography (silica gel, ethyl acetate/hexane=4:1). Optical purity of the product was measured using high resolution liquid chromatography equipped with a chiral column. The yield of the produced (S)-acetate was 93% and optical purity was 94% ee. The chiral acetate was hydrolyzed using a basic aqueous alcoholic solution and was converted to the corresponding chiral alcohol.

[α]²⁵_{D}=−92.6 (c=1.01, CHCl₃);

¹H NMR (300 MHz, CDCl₃, ppm) 7.29 (d, J=8.6 Hz, 2H), 6.87 (d, J=8.6 Hz, 2H), 5.86 (q, J=6.6 Hz, 1H), 3.80 (s, 3H), 2.28 (t, J=7.5 Hz, 2H), 1.68-1.57 (m, 2H), 1.51 (d, J=6.6 Hz, 3H), 0.91 (t, J=7.4 Hz, 3H).

### Example 4

The reaction procedure was performed in the same manner as in Example 3, except that 1-cyclohexylethanol was reacted in THF instead of 1-p-methoxyphenylethanol. The yield of the produced (S)-acetate was 92% and the optical purity was 98% ee.

[α]²⁵_{D}=−1.5 (c=0.98, CHCl₃);

¹H NMR (300 MHz, CDCl₃, ppm) 4.77-4.68 (m, 1H), 2.26 (t, J=7.4 Hz, 2H), 1.76-1.61 (m, 7H), 1.43-1.41 (m, 1H), 1.25-1.14 (m, 6H), 1.05-0.92 (m, 5H).

### Example 5

To a Schlenk flask, 10 mg of (η⁵-indanyl)RuCl(PPh₃)₂, 20 mg of stabilized subtilisin, 30 mg of 1-cyclohexylethanol, 75 mg of triethylamine, 75 μL of 4-chlorophenyl butyrate and 2 mL of dichloromethane were added and agitated in the presence of oxygen at 60° C. for three days. After termination of the reaction, catalyst was filtered, the obtained filtrated solution was concentrated and the product was separated using column chromatography (silica gel, ethyl acetate/hexane=4:1). Optical purity of the product was measured using high resolution liquid chromatography equipped with a chiral column. The yield of the produced (S)-acetate was 80% and the optical purity was 98% ee. The chiral acetate was hydrolyzed using basic aqueous alcoholic solution and was converted to the corresponding chiral alcohol.

[α]²⁵_{D}=−1.5 (c=0.98, CHCl₃);

¹H NMR (300 MHz, CDCl₃, ppm) 4.77-4.68 (m, 1H), 2.26 (t, J=7.4 Hz, 2H), 1.76-1.61 (m, 7H), 1.43-1.41 (m, 1H), 1.25-1.14 (m, 6H), 1.05-0.92 (m, 5H).

### Example 6

The reaction procedure was performed in the same manner as in Example 1, except that 1-phenyl-2-propanol was used instead of 1-phenylethanol. The yield of the produced (S)-acetate was 77% and the optical purity was 97% ee.

[α]²⁵_{D}=+12.1 (c=1.00, CHCl₃);

¹H NMR (300 MHz, CDCl₃, ppm) 7.31-7.18 (m, 5H), 5.13 (q, J=6.4 Hz, 1H), 2.92 (dd, J₁=13.6 Hz, J₂=6.8 Hz, 1H), 2.76 (dd, J₁=13.6 Hz, J₂ 6.4 Hz, 1H), 2.22 (t, J=7.4 Hz, 2H), 1.63-1.53 (m, 2H), 1.21 (d, J=6.3 Hz, 3H), 0.88 (t, J=7.4 Hz, 3H).

### Example 7

The reaction procedure was performed in the same manner as in Example 1, except that [(Ph₄C₅NHCHMe₂)Ru(CO)Cl]₂ was used instead of (Ph₄C₅NHCHMe₂)Ru(CO)₂Cl. The yield of the produced (S)-acetate was 82% and the optical purity was 70% ee.

[α]²⁵_{D}=−87.3 (c=1.01, CHCl₃);

¹H NMR (300 MHz, CDCl₃, ppm) 7.35-7.28 (m, 5H), 5.90 (q, J=6.6 Hz, 1H), 2.31 (t, J=7.4 Hz, 2H), 1.68-1.58 (m, 2H), 1.53 (d, J=6.6 Hz, 3H), 0.92 (t, J=7.4 Hz, 3H).

### Example 8

The reaction procedure was performed in the same manner as in Example 1, except that 1-phenyl-2-butanol was used instead of 1-phenylethanol. The yield of the produced (S)-acetate was 80% and the optical purity was 98% ee.

[α]²⁵_{D}=−5.6 (c=1.15, CHCl₃);

¹H NMR (300 MHz, CDCl₃, ppm) 7.30-7.15 (m, 5H), 4.98-4.92 (m, 1H), 2.68-2.59 (m, 2H), 2.26 (t, J=7.4 Hz, 2H), 1.94-1.78 (m, 2H), 1.70-1.62 (m, 2H), 1.24 (d, J=6.3 Hz, 3H), 0.95 (t, J=7.4 Hz, 3H).

### Example 9

The reaction procedure was performed in the same manner as in Example 1, except that 2-octanol was used instead of 1-phenylethanol. The yield of the produced (S)-acetate was 89% and the optical purity was 98% ee.

[α]²⁵_{D}=+5.7 (c=1.15, CHCl₃);

¹H NMR (300 MHz, CDCl₃, ppm) 4.95-4.85 (m, 1H), 2.27 (t, J=7.4 Hz, 2H), 1.68-1.58 (m, 2H), 1.56-1.37 (m, 2H), 1.27 (s, 8H), 1.19 (d, J=6.2 Hz, 3H), 0.94 (t, J=7.4 Hz, 3H), 0.87 (t, J=6.7 Hz, 3H).

### Example 10

To a Schlenk flask, 16 mg of [(p-cymene)RuCl₂]₂, 40 mg of stabilized subtilisin, 42 mg of 1-phenylethanol, 150 μL of 4-chlorophenyl butyrate and 1.5 mL of 1-butyl-3-methylimidazolium hexafluorophosphate ([BMIM]⁺PF₆⁻) were added and agitated at room temperature for five days. After termination of the reaction, catalyst was filtered, the obtained filtrated solution was extracted with chloroform. Extract was concentrated and the product was separated using column chromatography (silica gel, ethyl acetate/hexane=4:1). Optical purity of the product was measured using high resolution liquid chromatography equipped with a chiral column. The yield of the produced (S)-acetate was 98% and the optical purity was 89% ee. The chiral acetate was hydrolyzed using basic aqueous alcoholic solution and was converted to the corresponding chiral alcohol.

[α]²⁵_{D}=−87.3 (c=1.01, CHCl₃);

¹H NMR (300 MHz, CDCl₃, ppm) 7.35-7.28 (m, 5H), 5.90 (q, J=6.6 Hz, 1H), 2.31 (t, J=7.4 Hz, 2H), 1.68-1.58 (m, 2H), 1.53 (d, J=6.6 Hz, 3H), 0.92 (t, J=7.4 Hz, 3H).

### Example 11

The reaction procedure was performed in the same manner as in Example 1, except that 1-triphenylmethyloxy-2-propanol was used instead of 1-phenylethanol. The yield of the produced (S)-acetate was 71% and the optical purity was 99% ee.

[α]²⁵_{D}=+16.3 (c=1.0, CHCl₃, deacetylated product);

¹H NMR (300 MHz, CDCl₃, ppm) 7.46-7.24 (m, 15H), 5.17-5.12 (m, 1H), 3.16-3.08 (m, 2H), 2.35 (t, J=7.4 Hz, 2H), 1.72-1.65 (m, 2H), 1.21 (d, J=6.5 Hz, 3H), 0.94 (q, J=5.7 Hz, 3H).

### Example 12

The reaction procedure was performed in the same manner as in Example 1, except that 1-benzyloxy-3-chloro-2-propanol was used instead of 1-phenylethanol. The yield of the produced (S)-acetate was 80% and the optical purity was 98.5% ee.

¹H NMR (300 MHz, CDCl₃, ppm) 7.28-7.27 (m, 5H), 5.18 (q, J=5.2 Hz, 1H), 4.57-4.55 (m, 2H), 3.79-3.61 (m, 4H), 2.34 (t, J=6.5 Hz, 2H), 1.71-1.61 (m, 2H), 0.94 (q, J=5.7 Hz, 3H).

### Example 13

The reaction procedure was performed in the same manner as in Example 1, except that 1-phenyl-3-hydroxybutyne was used instead of 1-phenylethanol. The yield of the produced (S)-acetate was 90% and the optical purity was 95% ee.

[α]²⁵_{D}=−235.3 (c=0.7, CHCl₃);

¹H NMR (300 MHz, CDCl₃, ppm) 7.46-7.39 (m, 2H), 7.34-7.22 (m, 3H), 5,70 (q, J=6.7 Hz, 1H), 2.33 (t, J=7.4 Hz, 2H), 1.75-1.63 (m, 2H), 1.58 (d, J=6.7 Hz, 3H), 1.00 (t, J=7.4 Hz, 3H).

### Example 14

To a Schlenk flask, 5.9 mg of (η⁵-Ph₄C₄CO)₂H(μ-H)(CO)₄Ru₂, 16 mg of stabilized subtilisin, 62 mg of 3-(1-hydroxyethyl)phenyl butyrate, and 1 mL of toluene were added and agitated in the presence of argon gas at 60° C. for three days. After termination of the reaction, catalyst was filtered, the obtained filtrated solution was concentrated and the product was separated using column chromatography (silica gel, ethyl acetate/hexane=4:1). Optical purity of the product was measured using high resolution liquid chromatography equipped with a chiral column. The yield of the produced (S)-acetate was 94% and the optical purity was 99% ee. The chiral acetate was hydrolyzed using basic alcohol aqueous solution and was converted to the corresponding chiral alcohol.

[α]²⁵_{D}=−95.4 (c=1, CHCl₃);

¹H NMR (300 MHz, CDCl₃, ppm) 7.20 (t, J=7.9 Hz, 1H), 6.91 (d, J=7.6 Hz, 1H), 6.82 (s, 1H), 6.76 (dd, J=5.5 Hz, J₂=1.7 Hz, 1H), 5.83 (q, J=6.6 Hz, 1H), 2.32 (t, J=7.4 Hz, 2H), 1.70-1.62 (m, 2H), 1.51 (d, J=6.6 Hz, 3H), 0.94 (q, J=7.3 Hz, 3H).

### Example 15

To a Schlenk flask, 7.44 mg of (Ph₄C₅NHCHMe₂)Ru(CO)₂Cl, 7.5 mg of stabilized subtilisin, 47 mg of 1-p-chlorophenylethanol, 100 μL of 4-chlorophenyl butyrate and 1 mL of tetrahydrofuran were added and agitated at room temperature for three days. After termination of the reaction, catalyst was filtered, the obtained filtrated solution was concentrated and the product was separated using column chromatography (silica gel, ethyl acetate/hexane=4:1). Optical purity of the product was measured using high resolution liquid chromatography equipped with a chiral column. The yield of the produced (S)-acetate was 98% and the optical purity was 99% ee.

The reaction scheme to produce the chiral acetate is as follows:

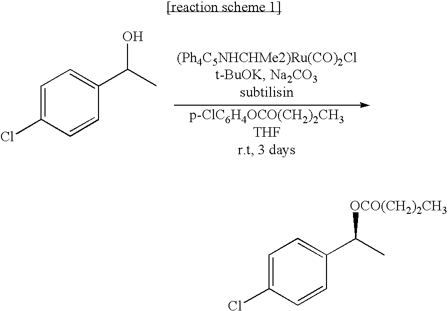

The produced chiral acetate was hydrolyzed using basic aqueous alcoholic solution and was converted to the corresponding chiral alcohol.

### Example 16

Chiral alcohol was obtained by performing the reaction procedure in the same manner as in Example 2, except that 41 mg of 1-phenyl-2-propanol was used instead of 43 mg of 1-p-chlorophenylethanol.

### Example 17

Chiral alcohol was obtained by performing the reaction procedure in the same manner as in Example 3, except that 41.6 mg of 1-(2-puryl)-butene-3-ol was used instead of 43 mg of 1-p-chlorophenylethanol.

### Example 18

Chiral alcohol was obtained by performing the reaction procedure in the same manner as in Example 17, except that 1-(cyclohexyl)-butene-3-ol was used instead of 1-(2-puryl)-butene-3-ol.

### Example 19

Chiral alcohol was obtained by performing the reaction procedure in the same manner as in Example 5, except that 34 mg of 1-indanol was used instead of 30 mg of 1-cyclohexylethanol.

### Example 20

Chiral alcohol was obtained by performing the reaction procedure in the same manner as in Example 1, except that 41.6 mg of 2-octanol was used Instead of 43 mg of 1-phenylethanol.

### Example 21

Chiral alcohol was obtained by performing the reaction procedure in the same manner as in Example 2, except that 2,5-hexandiol was used instead of 1-p-chlorophenylehanol.

### Example 22

Chiral alcohol was obtained by performing the reaction procedure in the same manner as in Example 10, except that 1,5-di(hydroxyethyl)pyridine was used instead of 1-phenylehanol.

### Example 23

Chiral alcohol was obtained by performing the reaction procedure in the same manner as in Example 2, except that methyl-4-phenyl-2-hydroxybutyrate was used Instead of 1-p-chlorophenylehanol.

### Example 24

Chiral alcohol was obtained by performing the reaction procedure in the same manner as in Example 2, except that 2-cyclohexyl-2-hydeoxyacetate was used instead of 1-p-chlorophenylehanol.

### Example 25

Chiral alcohol was obtained by performing the reaction procedure in the same manner as in Example 2, except that methyl 3-(4-methoxyphenyl)-3-hydroxypropionate was used instead of 1-p-chlorophenylehanol.

### Example 26

Chiral alcohol was obtained by performing the reaction procedure in the same manner as in Example 2, except that ethyl 3-phenyl-2-hydroxypropionate was used instead of 1-p-chlorophenylehanol.

### Example 27

Chiral alcohol was obtained by performing the reaction procedure in the same manner as in Example 2, except that t-butyl 5-hydroxyheptanoate was used instead of 1-p-chlorophenylehanol.

### Example 28

Chiral alcohol was obtained by performing the reaction procedure in the same manner as in Example 2, except that benzyl 3-hydroxybutyrate was used instead of 1-p-chlorophenylehanol.

### Example 29

Chiral alcohol was obtained by performing the reaction procedure in the same manner as in Example 2, except that 1-triphenylmethyloxy-2-butanol was used instead of 1-p-chlorophenylehanol.

### Example 30

Chiral alcohol was obtained by performing the reaction procedure in the same manner as in Example 2, except that 1-(5,9-dihydro-6,8-dioxabenzocyclohepene-7-yl-2-propanol was used instead of 1-p-chlorophenylehanol.

### Example 31

Chiral alcohol was obtained by performing the reaction procedure in the same manner as in Example 2, except that 1-t-butoxy-3-chloro-2-propanol was used instead of 1-p-chlorophenylehanol.

### Example 32

Chiral alcohol was obtained by performing the reaction procedure in the same manner as in Example 2, except that 1-phenyl-2-chloroethanol was used instead of 1-p-chlorophenylehanol.

### Example 33

Chiral alcohol was obtained by performing the reaction procedure in the same manner as in Example 2, except that 1-phenyl-2-azidoethanol was used Instead of 1-p-chlorophenylehanol.

### Example 34

Chiral alcohol was obtained by performing the reaction procedure in the same manner as in Example 2, except that 1-phenyl-2-cyanoethanol was used instead of 1-p-chlorophenylehanol.

### Example 35

To a Schlenk flask, 5.9 mg of (η⁵-Ph₄C₄CO)₂H(μ-H)(CO)₄Ru₂, 16 mg of stabilized subtilisin, 44 mg of 1-oxo-1,2,3,4-tetrahydronaphthalene, 39 μL of 4-chlorophenyl butyrate and 1 mL of toluene were added and agitated at 60° C. under 1 atm of hydrogen for three days. After termination of the reaction, catalyst was filtered, the obtained filtrated solution was concentrated and the product was separated using column chromatography (silica gel, ethyl acetate/hexane=4:1). Optical purity of the product was measured using high resolution liquid chromatography equipped with a chiral column. The produced chiral acetate was hydrolyzed using basic aqueous alcoholic solution and was converted to the corresponding chiral alcohol.

### Example 36

Chiral alcohol was obtained by performing the reaction procedure in the same manner as in Example 35, except that 1-phenyl-3-oxobutane was used instead of 1-oxo-1,2,3,4-tetrahydronaphthalene.

### Experimental Example 1

The reaction procedure was performed in the same manner as in Example 1, except that 1-phenylethanol was used as a substrate, 9.3 mg of (Ph₄C₅NHCHMe₂)Ru(CO)₂Cl, solvent, and acyl donor were used as described in Table 1.

TABLE 1

| | | | Optical |
|---|---|---|---|
| | | yield | purity |
| Solvent | Acyl donor | (%) | (% ee) |
| 2,2,4-trimethylpentane | p-chlorophenyl butyrate | 88 | 80 |
| Toluene | p-chlorophenyl butyrate | 86 | 79 |
| t-butyl methylether | p-chlorophenyl butyrate | 93 | 82 |
| methylene chloride | p-chlorophenyl butyrate | 91 | 87 |
| 1,4-dioxane | p-chlorophenyl butyrate | 98 | 84 |
| t-butanol | p-chlorophenyl butyrate | 94 | 91 |
| Tetrahydrofuran | p-chlorophenyl butyrate | 98 | 89 |
| Tetrahydrofuran | Isopropyl acetate | 22 | 71 |
| Tetrahydrofuran | 2,2,2-trifluoroethyl acetate | 60 | 52 |
| Tetrahydrofuran | 2,2,2-trifluoroethyl butyrate | 93 | 89 |

### INDUSTRIAL APPLICABILITY

According to the present invention, (S)-chiral alcohol can be synthesized with high optical purity and high yield by performing dynamic kinetic resolution with respect to an achiral substrate of ketone or a racemic alcohol by the combination of metal catalyst and protein hydrolysis enzyme. The (S)-chiral alcohol is an enantiomer of a chiral alcohol which can be obtained using lipase in conventional dynamic kinetic resolution method.

The method of synthesizing a chiral alcohol is variously applicable to obtain alcohols having various structures, compensating the conventional method using the lipase and can substitute for a conventional chemistry synthesis method or another biochemistry synthesis method.

Further, the (S)-chiral alcohol prepared according to the present invention can be used as an intermediate material of various chiral pharmaceuticals and fine chemicals.

What is claimed is:

1. A method of preparing (S)-chiral alcohol comprising:
(a) reacting in organic solvent a compound of the following chemical formula 1 as a starting material,
a racemization metal catalyst,
an acyl donor being capable of acylating an alcohol compound, and
a protein hydrolysis enzyme being capable of stimulating the enantioselective acylation of a racemic compound to obtain a chiral ester compound of chemical formula 3; and
(b) hydrolyzing the chiral ester compound of chemical formula 3 to obtain
(s)-chiral alcohol; 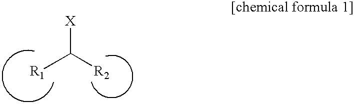 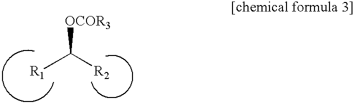
wherein X is —OH or ═O,R₁, R₂ and R₃ are independently substituted or unsubstituted C₁-C₁₅ alkyl, substituted or unsubstituted C₂-C₁₅ alkenyl, substituted or unsubstituted C₂-C₁₅, alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalky, wherein the R₁ group and the R₂ group can be linked together, and wherein a size of a circular arc indicates that the R₁ group is larger than the R₂ group.

2. The method of preparing (S)-chiral alcohol according to claim 1, further comprising adding a hydrogen donor in the (a) step when the starting material of chemical formula 1 comprises ketone such that X is ═O.

3. The method of preparing (S)-chiral alcohol according to claim 1:
wherein the starting material of chemical formula 1 is the compound of the following chemical formula 1a; 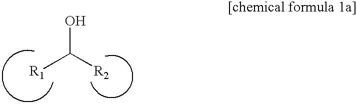
wherein R₁ and R₂ are independently substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalky; and
wherein R₁ and R₂ can be linked together.

4. The method of preparing (S)-chiral alcohol according to claim 3, further comprising:
obtaining an alcohol compound of chemical formula 1a by adding hydrogen donor to ketone compound of the following chemical formula 1b to reduce it; 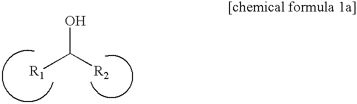 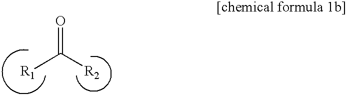
wherein R₁ and R₂ are independently substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalky; and
wherein R₁ and R₂ can be linked together.

5. The method of preparing (S)-chiral alcohol according to claim 1, further comprising:
adding hydrogen donor to reduce a ketone group in (a) step;
wherein the compound of chemical formula 1 comprises chemical formula 1b; 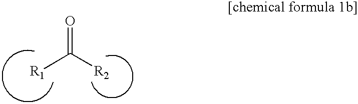
wherein R₁ and R₂ are independently substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalky; and
wherein R₁ and R₂ can be linked together.

6. The method of preparing (S)-chiral alcohol according to claim 1, wherein the (a) step reaction comprises a one-pot reaction and wherein the reaction is performed in one vessel.

7. The method of preparing (S)-chiral alcohol according to claim 1, wherein the metal catalyst comprises a ruthenium complex compound.

8. The method of preparing (S)-chiral alcohol according to claim 1, wherein the metal catalyst is selected from the group consisting of ruthenium complex compounds represented by the following chemical formulas 4 to 8: 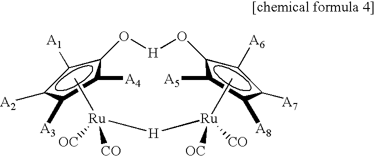 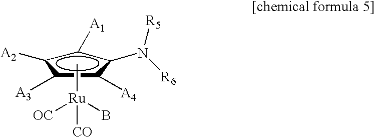 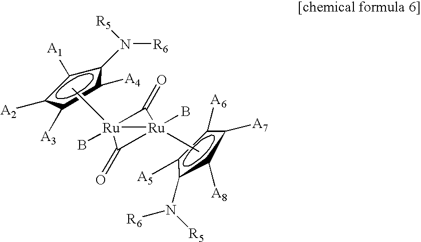 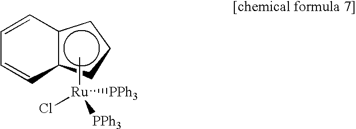 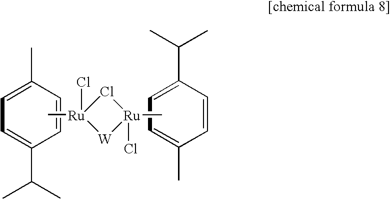
wherein A₁, A₂, A₃, A₄, A₅, A₆, A₇ and A₈ are independently hydrogen, substituted or unsubstituted C₁-C₁₀ alkyl, substituted or unsubstituted C₅-C₁₈ aryl, or substituted or unsubstituted C₂-C₂₀ heterocycle;
wherein R₅ and R₆ are independently hydrogen, substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₁-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalkyl;
wherein B comprises a substituent selected from the group consisting of hydrogen, carbonyl, halogen and trifluoromethanesulfonate or there is no substituent in B site; and
wherein W is hydrogen or a halogen.

9. The method of preparing (S)-chiral alcohol according to claim 2, wherein the acyl donor comprises 2,4-dimethyl-3-pentanol, 2,6-dimethyl-4-heptanol, formic acid, or hydrogen.

10. The method of preparing (S)-chiral alcohol according to claim 1, wherein the acyl donor is linked to the R₁ group or the R₂ group of the chemical formula 1.

11. The method of preparing (S)-chiral alcohol according to claim 10, wherein the acyl donor is a substituent including —OCO—R₃ terminal group linked to the R₁ or R₂ of the chemical formula 1.

12. The method of preparing (S)-chiral alcohol according to claim 1, wherein the acyl donor is the compound of the chemical formula 2; and
[chemical formula 2] 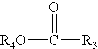
wherein R₃ and R₄ are independently substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalkyl.

13. The method of preparing (S)-chiral alcohol according to claim 1, wherein the protein hydrolysis enzyme is selected from the group consisting of stabilized or fixed subtilisin, chymotrypsin, papain, protease from Aspergillus orygae, protease from Aspergillus melleus, protease from Streptomyces griseus, and protease from Bacillus stearothemophilus.

14. The method of preparing (S)-chiral alcohol according to claim 1, wherein the protein hydrolysis enzyme is subtilisin.

15. The method of preparing (S)-chiral alcohol according to claim 1, wherein the organic solvent is benzene, toluene, C₅-C₁₀ alkane, C₅-C₁₀ cycloalkane, tetrahydrofuran, dioxane, C₂-C₁₀ dialkylether, C₃-C₁₀ alkylate, C₂-C₁₀ cyanoalkane, C₃-C₁₀ dialkyl ketone, dichloromethane, chloroform, carbon tetrachloride, C₄-C₁₀ tertiary alcohol, or a room temperature ionic liquid.

16. The method of preparing (S)-chiral alcohol according to claim 1, wherein the reaction temperature in (a) step is room temperature to 80° C.

17. A (S)-chiral alcohol prepared according to claim 1.

18. A method of preparing (S)-chiral ester comprising:
reacting in organic solvent the compound of the following chemical formula 1 as a starting material,
a racemization metal catalyst,
an acyl donor being capable of acylating an alcohol compound, and
a protein hydrolysis enzyme being capable of stimulating the enantioselective acylation of a racemic compound to obtain a chiral ester compound of chemical formula 3. 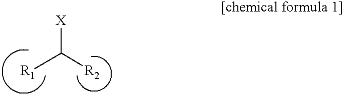 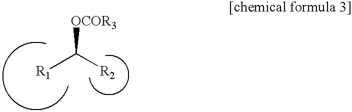
wherein R₁ and R₂ are independently substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalky, and R₁ and R₂ can be linked together; and
wherein a size of a circular arc indicates that the R₁ group is larger than the R₂ group.

19. The method of preparing (S)-chiral ester according to claim 18, further comprising adding a hydrogen donor in the (a) step and when the starting material comprises ketone where X═O.

20. A (S)-chiral ester of the following chemical formula 3 prepared according to claim 18; 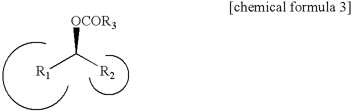
wherein R₁, R₂ and R₃ are independently substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₅ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalkyl;
wherein the R₁ group and the R₂ group can be linked together; and
wherein a size of a circular arc indicates that the R₁ group is larger than the R₂ group.

21. A method of preparing (S)-chiral alcohol of the following chemical formula 1 comprising:
hydrolyzing the chiral ester of the chemical formula 3 prepared according to claim 18. 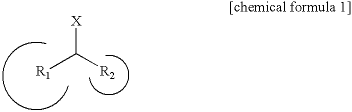 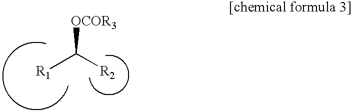
wherein X—OH or ═O,
wherein R₁, R₂ and R₃ are independently substituted or unsubstituted C₁-C₁₅ alkyl, substituted or unsubstituted C₂-C₁₅ alkenyl, substituted or unsubstituted C₂-C₁₅ alkynyl, substituted or unsubstituted C₅-C₁₈ aryl, substituted or unsubstituted C₆-C₁₈ arylalkyl, substituted or unsubstituted C₂-C₂₀ heterocycle, substituted or unsubstituted C₃-C₂₀ heteroarylalkyl, substituted or unsubstituted C₃-C₁₆ cycloalkyl, substituted or unsubstituted C₃-C₁₅ cycloalkenyl, substituted or unsubstituted C₆-C₁₅ cycloalkynyl, or substituted or unsubstituted C₃-C₂₀ heterocycloalkyl;
wherein R₁ and R₂ can be linked together; and
wherein a size of a circular arc indicates that the R₁ group is larger than the R₂ group.
